(19) United States
(12) Patent Application Publication
(10) Pub. No.: US 2023/0038483 A1
(43) Pub. Date: Feb. 9, 2023

(54) AN ADHESIVE BODY DRESSING AND METHOD FOR MANUFACTURING AN ADHESIVE BODY DRESSING

(71) Applicant: DERMECO OY, Helsinki (FI)

(72) Inventors: Roosa KIVIVUORI, Helsinki (FI); Heini ISOAHO, Helsinki (FI)

(21) Appl. No.: 17/792,196

(22) PCT Filed: Jan. 15, 2021
(86) PCT No.: PCT/FI2021/050022

§ 371 Date: Jul. 12, 2022

(30) Foreign Application Priority Data
Jan. 17, 2020 (FI) 20205047

(51) Int. Cl. A61L 15/22; A61L 15/58; A61L 15/60; A61L 15/62

(52) U.S. Cl. CPC A61L 15/225; A61L 15/585; A61L 15/60; A61L 15/62

(57) ABSTRACT

An adhesive body dressing includes a hydrocolloid adhesive layer (H) and a backing layer (B) provided on a first side of the hydrocolloid adhesive layer (H). The backing layer (B) is made of biodegradable polymer material.

### FIELD OF THE INVENTION

The present invention relates to an adhesive body dressing and more particularly to an adhesive body dressing according to the preamble of claim 1. The present invention also relates to a method for manufacturing an adhesive body dressing and more particularly to a method according to preamble of claim 14

### BACKGROUND OF THE INVENTION

Adhesive hydrocolloid dressings are generally known for many different uses as polymer dressings. These adhesive hydrocolloid dressings are used as body or skin dressings for improving or preventing any skin related medical or esthetical concerns such as wounds, blisters, cold sores, acne, corns, cracked skin, burns, pressure ulcers, eczema, scarring or stretch marks. dressings for dermal ulcers, burns and other exudative wounds.

The adhesive hydrocolloid dressings are used for certain time depending on the purpose of use. After use, the adhesive hydrocolloid dressings are removed from the body and skin and discarded. The hydrocolloid material in the adhesive hydrocolloid dressing absorbs moisture from the skin or body creating advantageous healing conditions for a wound or advantageous treatment conditions for skin. At the same time the hydrocolloid adhesive composition is susceptibility to breakdown and detachment form the skin upon swelling due to absorbed moisture. This together with external wearing limit the use time of the adhesive hydrocolloid dressing.

Due to the short-term use of the adhesive hydrocolloid dressings as wound dressings and dressings for blisters or the like, they are often removed and discarded in acute situations. Thus, the adhesive hydrocolloid dressings may often end nature and landfills where they may remain causing long-term waste problems.

### BRIEF DESCRIPTION OF THE INVENTION

An object of the present invention is to provide an adhesive body dressing and the method for manufacturing an adhesive body dressing so as to solve or at least alleviate the prior art problems.

The objects of the invention are achieved by an adhesive dressing which is characterized by what is stated in independent claim 1. The objects of the invention are further achieved by a method which is characterized by what is stated in independent claim 14. The objects of the invention are also achieved by a use is characterized by what is stated in independent claim 16.

The preferred embodiments of the invention are disclosed in the dependent claims.

The invention is based on the idea of providing an adhesive body dressing comprising a hydrocolloid adhesive layer and a backing layer provided on a first side of the hydrocolloid adhesive layer. According to the present invention the backing layer and the hydrocolloid adhesive layer are made of biodegradable polymer materials.

Durability of the hydrocolloid adhesive compositions due to their susceptibility to breakdown upon exposure to excessive moisture or body fluids is enhanced by providing a backing layer on one side of the adhesive hydrocolloid layer. This backing layer is made from biodegradable polymer material providing structural support and durability for the hydrocolloid adhesive layer. Hydrocolloid adhesive layers and compositions are usually biodegradable and thus providing the backing layer from a biodegradable polymer material enables proving the whole adhesive hydrocolloid body dressing as a biodegradable dressing. Thus, the adhesive hydrocolloid body dressing according to the present invention under goes a natural degradation in nature or in landfills preventing accumulation of waste materials.

In the context of this application term “biodegradable” means that material or substance can be completely converted into water, CO2, and biomass through the action of microorganisms such as fungi and bacteria. This property is not dependent on the origin of the raw materials, but only on the chemical composition of the polymers. Further, biodegradability of polymers is appropriately described by test and assessment methods in international standards. Industrial composting is today a well-established disposal process for organic waste materials. One test of biodegradability is the controlled composting test according to ISO 14855.

Additionally, ISO 17088, the European standard 13432 and the American standard ASTM D 6400 define basic requirements for packaging and packaging materials to be considered as biodegradable and compostable in industrial composting facilities.

In the context of this application biodegradable polymer material and term biodegradable mean a polymer based material which fulfils requirements for a biodegradable and compostable polymer material according to one or more of the following standards: ISO 17088:2012, ISO 14855-1, ISO 14855-2, ISO 16929, ISO 20200, EN 13432:2000, ASTM D5338, ASTM D6400, and OECD Guidelines for the Testing of Chemicals—Guideline 208: Terrestrial Plants, Growth Test.

In one embodiment, the hydrocolloid adhesive layer comprises a hydrocolloid adhesive material layer. The backing layer is provided on a first side of the hydrocolloid adhesive material layer.

In another embodiment, the hydrocolloid adhesive layer comprises a first hydrocolloid adhesive material layer and a second hydrocolloid adhesive material layer. The first hydrocolloid adhesive material layer provided between the backing layer and the second hydrocolloid adhesive material layer. The first and second hydrocolloid adhesive material layers are superposed in the adhesive body dressing. The first hydrocolloid adhesive material layer is provided on a second side of the second hydrocolloid adhesive material layer and the second hydrocolloid adhesive material layer is provided on a second side of the backing layer.

In one embodiment, the hydrocolloid adhesive layer comprises biodegradable polymer based material.

In another embodiment, the hydrocolloid adhesive material layer is made of biodegradable polymer material.

In a further embodiment, the first hydrocolloid adhesive material layer and the second hydrocolloid adhesive material layer are made of biodegradable polymer materials.

According to the above mentioned, the hydrocolloid adhesive layer and the backing layer are both made of biodegradable polymer material such that the whole adhesive body dressing is biodegradable.

In one embodiment, the backing layer comprises a surface backing material layer provided on the first side of the hydrocolloid adhesive layer. This provides simple constructions with single backing material layer.

In another embodiment, the backing layer comprises a surface backing material layer and an intermediate backing material layer, the intermediate backing material layer is provided between the surface backing material layer and the hydrocolloid adhesive layer. This provides enhanced elasticity and durability for the adhesive body dressing. This may also enhance the attachment of the surface backing material layer to the hydrocolloid adhesive layer.

In one embodiment, the backing layer or the surface backing material layer comprises biodegradable agro-polymers.

The backing layer or the surface backing material layer is made of biodegradable agro-polymers.

In another embodiment, the backing layer or the surface backing material layer comprises biodegradable biopolymers.

The backing layer or the surface backing material layer is made of biodegradable biopolymers.

In a further embodiment, the backing layer or the surface backing material layer comprises biodegradable agro-polymers and biodegradable biopolymers.

The backing layer or the surface backing material layer is made of biodegradable agro-polymers and biodegradable biopolymers.

In one embodiment, the backing layer or the surface backing material layer comprises enzymatically degradable polymers.

The backing layer or the surface backing material layer is made of enzymatically degradable polymers.

In another embodiment, the backing layer or the surface backing material layer comprises hydrolytically degradable polymers.

The backing layer or the surface backing material layer is made of hydrolytically degradable polymers.

In a further embodiment, the backing layer or the surface backing material layer comprises enzymatically degradable polymers and hydrolytically degradable polymers.

The backing layer or the surface backing material layer is made of enzymatically degradable polymers and hydrolytically degradable polymers.

Hydrolytically degradable polymers are advantageous to undergo degradation in nature due to moisture or humid conditions.

The enzymatically biodegradable polymers are advantageous under suitable enzyme conditions.

In one embodiment, the backing layer or the surface backing material layer comprises biodegradable agro-polymers.

The backing layer or the surface backing material layer is made of biodegradable agro-polymers.

In another embodiment, the backing layer or the surface backing material layer comprises biodegradable agro-polymers, the biodegradable agro-polymers comprising one or more of the following: polysaccharides, proteins and lignins.

The backing layer or the surface backing material layer is made of one or more of the following polysaccharides, proteins and lignins.

Agro-polymers are biomass products and from natural biomass sources. Agro-polymers are thus especially environmentally friendly.

Agro-polymers are also enzymatically degradable polymers.

In one embodiment, the backing layer or the surface backing material layer comprises biodegradable biopolyesters.

The backing layer or the surface backing material layer is made of biodegradable biopolyesters.

In another embodiment, the backing layer or the surface backing material layer comprises biodegradable biopolyesters, the biodegradable biopolyesters comprising one or more of the following: polyhydroxyalkanoates, polylactides, polyglycolide, polycaprolactones, polyesteramides, polyurethane, aliphatic copolyesters and aromatic copolyesters.

The backing layer or the surface backing material layer is made of biodegradable biopolyesters, the biodegradable biopolyesters comprising one or more of the following: polyhydroxyalkanoates, polylactides, polyglycolide, polycaprolactones, polyesteramides, polyurethane, aliphatic copolyesters and aromatic copolyesters.

The biodegradable biopolyesters are hydrolytically degradable polymers.

The biodegradable polyesters are obtained from micro-organisms by extraction, from biotechnology by synthesis from bio-derived monomers, or from petrochemical products by synthesis form synthetic monomers. Thus, residual or recycled materials may be used for providing the biodegradable polyesters.

In one embodiment, the backing layer or the surface backing material layer is a film layer.

In another embodiment, the backing layer or the surface backing material layer is a foam layer.

In one embodiment, the intermediate backing material layer is an intermediate elastomer layer. The elastomer layer provides elasticity to the backing structure and also enabled securing the surface backing layer to the hydrocolloid adhesive layer in elastic manner such that the backing layer and the surface backing layer remain attached to the hydrocolloid adhesive layer during use and under stress.

In one embodiment, the intermediate backing material layer comprises one or more of the following:
biodegradable agro-polymers;
biodegradable agro-polymers, the biodegradable agro-polymers comprising one or more of the following: polysaccharides, proteins and lignins;
biodegradable biopolyesters; and
biodegradable biopolyesters, the biodegradable biopolyesters comprising one or more of the following: polyhydroxyalkanoates, polylactides, polyglycolide, polycaprolactones, polyesteramides, polyurethane, aliphatic copolyesters and aromatic copolyesters.

In one embodiment, the intermediate backing material layer is made of one or more of the following:
biodegradable agro-polymers;
biodegradable agro-polymers, the biodegradable agro-polymers comprising one or more of the following: polysaccharides, proteins and lignins;
biodegradable biopolyesters; and
biodegradable biopolyesters, the biodegradable biopolyesters comprising one or more of the following: polyhydroxyalkanoates, polylactides, polyglycolide, polycaprolactones, polyesteramides, polyurethane, aliphatic copolyesters and aromatic copolyesters.

Accordingly, the intermediate backing material layer is also provided biodegradable making the whole backing layer biodegradable.

In one embodiment, the backing layer has a thickness between 0.01-to 0.1 mm.

In another embodiment, the backing layer has preferably a thickness between 0,02-0.05 mm.

The relatively small thickness of the backing layer provides necessary surface layer for the hydrocolloid adhesive layer without making the adhesive body dressing unnecessary thick.

In one embodiment, the hydrocolloid adhesive layer has thickness between 0,3 to 3 mm.

In another embodiment, the hydrocolloid adhesive layer has thickness between 0.5 to 2 mm.

In further embodiments, the backing layer has a thickness between 0,01-to 0.2 mm.

Also in further embodiments, the hydrocolloid adhesive layer has a thickness between 0.1 to 10 mm, or between 0.1 to 5 mm.

The thickness of the hydrocolloid adhesive layer is provided with a material thickness such that adequate material volume is achieved for adsorption of body fluids during use of the adhesive body dressing.

In one embodiment, the adhesive body dressing comprises a first removable covering layer provided on a second side of the hydrocolloid adhesive layer. The first removable covering layer protects the adhesive surface of the hydrocolloid adhesive layer or the adhesive body dressing before placing the adhesive body dressing on skin of a user. Thus, the adhesive surface of the adhesive body dressing which is to be placed against the skin of the user may be kept clean without deterioration before use.

The first removable covering layer may also comprise or made of biodegradable polymer material. The biodegradable polymer material of the first removable covering layer may be or comprise one of the biodegradable polymer materials mentioned above in connection with the backing layer.

The adhesive body dressing may be for example a plaster, a patch or mask or the like.

The present invention also relates to a method for manufacturing an adhesive body dressing having a hydrocolloid adhesive layer and a backing layer.

The method comprises forming a backing layer of a biodegradable polymer material, and providing an adhesive hydrocolloid layer on the backing layer, the adhesive hydrocolloid layer being made of biodegradable material.

Accordingly, an adhesive body dressing which is fully biodegradable is achieved.

In one embodiment, the method comprises the method comprises manufacturing an adhesive body dressing as described above.

The present invention further relates to use of a biodegradable material for providing a backing layer of an adhesive body dressing comprising a hydrocolloid adhesive layer, the backing layer being provided on a first side of the hydrocolloid adhesive layer. The biodegradable material being one of the above identified biodegradable materials. Using a biodegradable polymer material for forming the backing layer enables providing a fully biodegradable adhesive body dressing.

An advantage of the invention is that the adhesive body dressing having a hydrocolloid adhesive layer is provided fully biodegradable. Thus, the adhesive body dressing degrades in nature diminishing waste problem and providing environmentally friendly the adhesive body dressing.

### BRIEF DESCRIPTION OF THE DRAWINGS

The invention is described in detail by means of specific embodiments with reference to the enclosed drawings, in which

FIG. 1 shows schematically an upper view of an adhesive body dressing;

FIG. 2 shows schematically a bottom view of an adhesive body dressing;

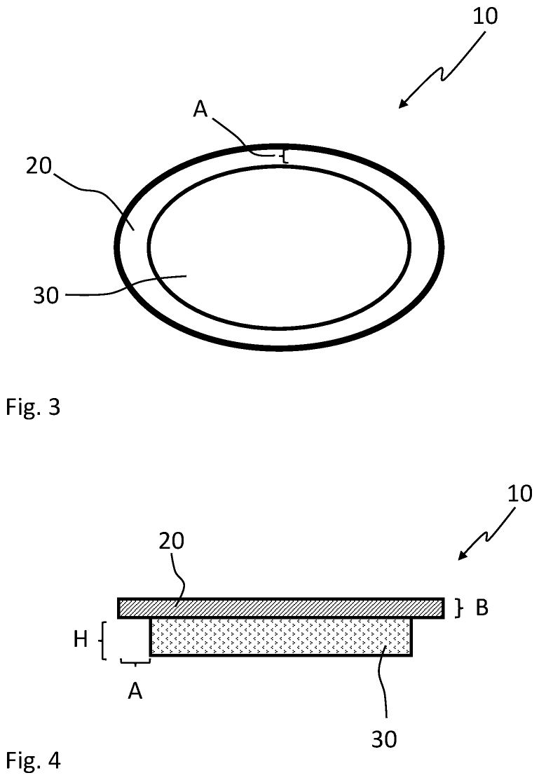

FIGS. 3 and 4 show schematically general structure of one embodiment of an adhesive body dressing according to the present invention;

FIGS. 5 to 12 show schematically different embodiments of an adhesive body dressing according to the present invention; and

FIG. 13 shows schematically a further embodiment of an adhesive body dressing according to the present invention.

### DETAILED DESCRIPTION OF THE INVENTION

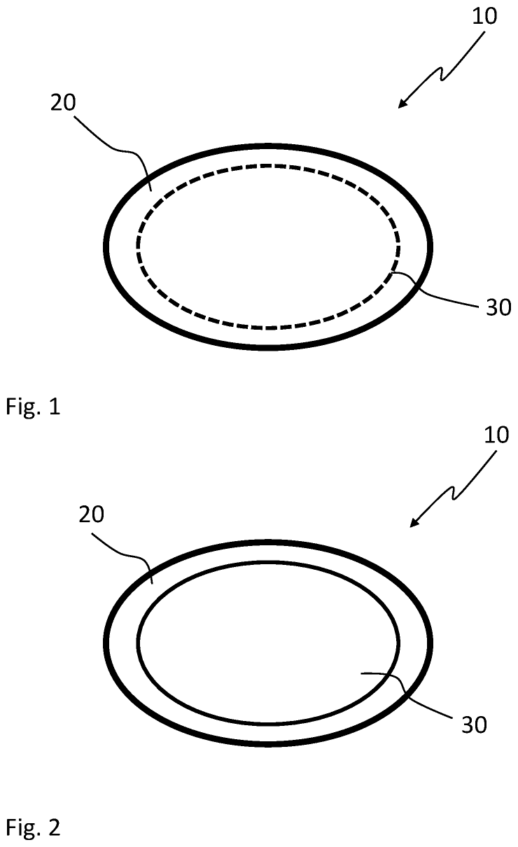

FIG. 1 shows schematically a top view of one embodiment of an adhesive body dressing 10. The adhesive body dressing 10 comprises a backing material layer 20 on top surface of the adhesive body dressing 10. The adhesive body dressing further comprises a hydrocolloid adhesive material layer 30 on bottom surface of the adhesive body dressing 10.

It should be noted, that the size and shape of the adhesive body dressing 10 of the present invention may vary and the present invention is not restricted to any particular shape or size.

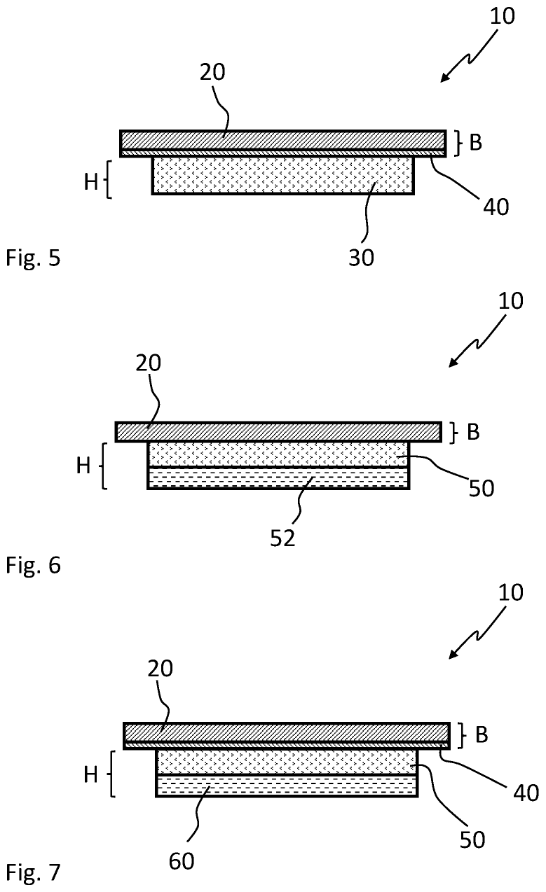

Usually the backing material layer 20 is larger than the hydrocolloid adhesive material layer 30. Thus, the surface area of the backing material layer 20 is larger than the surface area of the hydrocolloid adhesive material layer 30, as shown in FIG. 3. Accordingly, the hydrocolloid adhesive material layer 30 is provided at peripheral distance A from the peripheral edges of the backing material layer 20. The backing material layer 20 extends beyond the hydrocolloid adhesive material layer 30 or the peripheral edges of the hydrocolloid adhesive material layer 30 such that the peripheral area A is provided. This enables the hydrocolloid adhesive material layer 30 to be protected and not to be exposed during use of the adhesive body dressing. The hydrocolloid adhesive material layer 30 remains covered between the backing material layer 20 and the skin of the user and against the skin of the user.

FIG. 4 shows schematically a cross-sectional view of an adhesive body dressing 10. The adhesive body dressing 10 comprises a backing layer B and hydrocolloid adhesive layer H. The backing layer B and the hydrocolloid adhesive layer H are superposed such that the backing layer B is provided on a first side of the hydrocolloid adhesive layer H. Thus, the backing layer B and the hydrocolloid adhesive layer H are superposed in the structure of the adhesive body dressing 10. Second side of the hydrocolloid adhesive layer H is arranged to be placed against the skin of the user.

As shown in FIG. 4, the backing layer B extends beyond the hydrocolloid adhesive layer H or the peripheral edges of the hydrocolloid adhesive layer H such that the peripheral area A is provided around the hydrocolloid adhesive layer H.

In some other alternative embodiments, the backing layer B and the hydrocolloid adhesive layer H have substantially same or identical surface area. In that case, the peripheral area A is omitted or there is no peripheral area A. Thus, the peripheral edges of the backing layer B and the peripheral edges of the hydrocolloid adhesive layer are aligned such that the surface area of the backing layer B is substantially same as the surface area of the hydrocolloid adhesive layer H.

In the context of this application hydrocolloid adhesive layer comprises one or more layers of the hydrocolloid adhesive material. Hydrocolloid material or hydrocolloid adhesive material or hydrocolloid dressing material refers to a flexible sheet or wafer which contains material that forms a gel in the presence of water. Hydrocolloid dressing material may be adhered to the skin without the use of adhesives or separate taping, as the hydrocolloid adhesive material or hydrocolloid dressing material is self-adhesive or comprises self-adhesive properties.

The active surface of a hydrocolloid dressing or hydrocolloid layer H may be coated with a cross-linked adhesive mass containing a dispersion of gelatin, pectin and carboxy-methylcellulose (e.g., sodium carboxymethylcellulose (NaCMC) together with other polymers and adhesives. Suitably the hydrocolloid is combined with elastomers and adhesives and applied to the backing layer B. In contact with a fluid (e.g., a wound exudate, pus, oil, blood, etc.), the polysaccharides and other polymers of the hydrocolloid material of the hydrocolloid adhesive layer H absorb water and swell, forming a gel which is held within the structure of the adhesive matrix. The gel which is formed as a result of the absorption of fluid is not mobile and free running but held within the structure of the adhesive matrix. Suitably, a hydrocolloid dressing is waterproof. In some embodiments, hydrocolloid adhesive material comprise a hydrophobic pressure sensitive adhesive (e.g., butyl rubber or a styrene-isoprene-styrene (SIS) block copolymer) compounded with hydrophilic particulates.

In the present invention, the backing layer B comprises or is made of biodegradable polymer material.

In some embodiments, the backing layer B comprises or is made of biodegradable agro-polymers and/or biodegradable biopolyesters.

The biodegradable agro-polymers comprise polysaccharides, proteins and lignins.

The polysaccharides comprise starches such as wheat, potatoes and maize, ligno-cellulose products such as wood and straws, and other such as pectins, chitosan/chitin and gums.

The proteins and lignins comprise animal proteins such as casein, whey and collagen/gelatin, and plant proteins such as zein, soya and gluten. The biodegradable biopolyesters comprise polyhydroxyalkanoates, polylactides, polyglycolide, polycaprolactones, polyesteramides, polyurethane, aliphatic copolyesters and aromatic copolyesters.

The polyhydroxyalkanoates comprise such as poly(hydroxybutyrate) and poly(hydroxybutyrate cohydroxyvalerate). The polyhydroxyalkanoates are obtained from micro-organisms by extraction.

The polylactides comprise such as polylactic acid. The polylactides are obtained from biotechnology by conventional synthesis from bio-derived monomers.

The polycaprolactones, polyesteramides, aliphatic copolyesters and aromatic copolyesters are obtained by conventional synthesis from synthetic monomers.

The agro-polymers are enzymatically degradable polymers. The biopolyesters are hydrolytically degradable polymers. Thus agro-polymers may withstand water and humid conditions better than the biopolyesters. On the other hand, the biopolyesters may be preferable in conditions of low moisture or humidity.

The backing layer B may comprise one or more material layers which are made of or comprise same or different biodegradable materials.

In the embodiment of FIG. 4, the backing layer B comprise one material layer 20. In this embodiment, the backing layer B comprises a surface backing material layer 20 provided on the hydrocolloid adhesive layer H. The surface backing material layer 20 forms the outer and top surface of the adhesive body dressing. The surface backing material layer 20 is made of or comprises one or more biodegradable polymer materials.

The surface backing layer 20 comprises a top surface 24 and a bottom surface 22. The top surface 24 of the surface backing material layer 20 forms the top surface of the adhesive body dressing 10. Top surface 24 of the surface backing material layer 20 is preferably smooth and non-adhesive.

The bottom surface 22 of the surface backing material layer 20 is arranged towards or against the hydrocolloid adhesive layer H, as shown in FIG. 8. The bottom surface 22 of the surface backing material layer 20 or the surface backing material layer 20 may be adhesive or self-adhesive for attaching to the hydrocolloid adhesive layer H and/or to the skin at the peripheral area A. Alternatively, the bottom surface 22 of the surface backing material layer 20 may be provided with additional adhesive layer (not shown). The additional adhesive layer is preferably made of biodegradable adhesive material.

The surface backing material layer 20 may be a film layer or a foam layer.

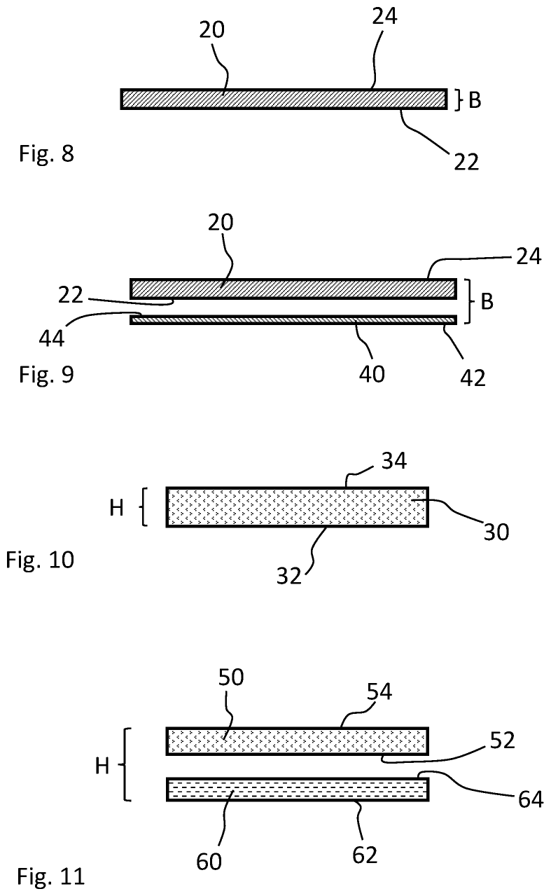

FIG. 5 shows one embodiment in which the backing layer B comprises the surface backing material layer 20 and an intermediate backing material layer 40. The intermediate backing material layer 40 is provided between the surface backing material layer 20 and the hydrocolloid adhesive layer H.

The intermediate backing material layer 40 is arranged against the bottom surface 22 of the surface backing material layer 20 and the upper surface of the hydrocolloid adhesive layer H.

In some embodiments, the intermediate backing material layer 40 is an intermediate elastomer layer.

In some embodiments, the intermediate backing material layer 40 comprises or is made of biodegradable agro-polymers and/or biodegradable biopolyesters.

The intermediate backing material layer 40 comprises an intermediate top surface 44 and an intermediate bottom surface 42, as shown in FIG. 9. The intermediate top surface 44 of the intermediate backing material layer 40 is arranged towards or against the surface backing material layer 20 and/or the bottom surface 22 thereof. The intermediate bottom surface 42 of the intermediate backing material layer 40 is arranged towards or against the hydrocolloid adhesive layer H.

The surface backing material layer 20 or the intermediate backing material layer 40 may be adhesive or self-adhesive for surface backing material layer 20 and the intermediate backing material layer 40 together. Alternatively, an additional adhesive layer may be provided between the bottom surface 22 of the surface backing material layer 20 and the intermediate top surface 44 of the intermediate backing material layer 40. The additional adhesive layer is preferably made of biodegradable adhesive material.

The intermediate bottom surface 42 of the intermediate backing material layer 40 is arranged towards or against the hydrocolloid adhesive layer H, as shown in FIG. 9. The intermediate bottom surface 42 of the intermediate backing material layer 40 or the intermediate backing material layer 40 may be adhesive or self-adhesive for attaching to the hydrocolloid adhesive layer H and/or to the skin at the peripheral area A. Alternatively, the intermediate bottom surface 42 of the intermediate backing material layer 40 may be provided with additional adhesive layer (not shown). The additional adhesive layer is preferably made of biodegradable adhesive material.

The hydrocolloid adhesive layer H may comprise one or more hydrocolloid material layers which are made of or comprise same or different hydrocolloid materials.

The one or more hydrocolloid material layers are preferably made of or comprise one or more biodegradable polymer based materials.

In the embodiment of FIGS. 4 and 10, the hydrocolloid adhesive layer H comprise only one hydrocolloid adhesive material layer 30.

The hydrocolloid adhesive material layer 30 comprises a hydrocolloid top surface 34 and a hydrocolloid bottom surface 32, as shown in FIG. 10. The hydrocolloid bottom surface 32 of the hydrocolloid adhesive material layer 30 forms bottom surface of the adhesive body dressing 10 and an active surface of the adhesive body dressing 10 which is to be placed against the skin of the user.

The hydrocolloid adhesive material layer 30 and the hydrocolloid bottom surface 32 thereof is usually self-adhesive for attachment to skin of the user. Alternatively, the hydrocolloid bottom surface 32 of the hydrocolloid adhesive material layer 30 may be provided with additional adhesive layer (not shown). The additional adhesive layer is preferably made of biodegradable adhesive material.

The hydrocolloid top surface 34 of the hydrocolloid adhesive material layer 30 is arranged towards or against the backing material layer B, or the surface backing material layer 20 and/or the bottom surface 22 thereof, or the intermediate backing material layer 40 and/or the intermediate bottom surface 42 thereof.

The hydrocolloid adhesive material layer 30 may be adhesive or self-adhesive for attaching the hydrocolloid adhesive material layer 30 and the backing layer B together. Alternatively, an additional adhesive layer may be provided between the hydrocolloid top surface 34 backing layer B. The additional adhesive layer is preferably made of biodegradable adhesive material.

FIGS. 6, 7 and 11 show an alternative embodiment in which the hydrocolloid adhesive layer H comprises a first hydrocolloid adhesive material layer 50 and a second hydrocolloid adhesive material layer 60. The first hydrocolloid adhesive material layer 50 is provided between the backing layer B and the second hydrocolloid adhesive material layer 60.

The first hydrocolloid adhesive material layer 50 and the second hydrocolloid adhesive material layer 60 are preferably made of or comprise biodegradable polymer materials.

The first hydrocolloid adhesive material layer 50 comprises a first hydrocolloid top surface 54 and a first hydrocolloid bottom surface 52, as shown in FIG. 11. The second hydrocolloid adhesive material layer 60 comprises a second hydrocolloid top surface 64 and a second hydrocolloid bottom surface 62.

The first hydrocolloid top surface 54 forms the hydrocolloid top surface 34 and the second hydrocolloid bottom surface 62 forms the bottom surface of the active surface of the adhesive body dressing 10 to be placed against skin of the user.

The first hydrocolloid bottom surface 52 and the second hydrocolloid top surface 64 are arranged towards or against each other.

It should be noted that the hydrocolloid adhesive layer H may comprise one or more hydrocolloid adhesive material layers 30, 50, 60 made of same or different material or hydrocolloid materials or comprises same or different hydrocolloid substances.

Similarly, the backing layer may comprise one or more backing material layers 20, 40 made of or comprising same or different biodegradable polymer materials.

FIG. 12 shows an embodiment of the adhesive body dressing 10 in which a first removable covering layer 80 provided on a second side of the hydrocolloid adhesive layer H or on the bottom or active bottom surface of the adhesive body dressing 10. The first covering layer 80 may be a conventional release liner which is used to protect the exposed active surface of the hydrocolloid adhesive layer H, and optionally the exposed portion of the backing layer B, the peripheral area A. The first covering layer 80 is removed just before applying the adhesive body dressing 10 to skin of the user.

A second removable covering layer 70 is provided the top surface of the backing layer B or the top surface 24 of the backing material layer 20 for preventing wrinkling, curling or deteriorating of the edges of backing layer B. The second covering layer 70 may support the exposed periphery of backing layer B during application of the adhesive body dressing 10 to skin of the user. Once the adhesive body dressing 10 is in place on the skin, the second covering layer is removed.

The first and/or second removable covering layers 80, 70 may also comprise or made of biodegradable polymer material. The biodegradable polymer material of the first and/or second removable covering layers 80, 70 may be made of or comprise one of the biodegradable polymer materials mentioned above in connection with the backing layer B.

Alternatively, the first and/or second removable covering layer may be made of biodegradable paper-based material or some biodegradable material.

The backing layer B may have a preferably thickness between 0.01-to 0.1 mm, preferably the backing layer B has preferably a thickness between 0,02-0.05 mm.

The hydrocolloid adhesive layer H may have thickness between 0,3 to 3 mm, or preferably the hydrocolloid adhesive layer H has thickness between 0.5 to 2 mm.

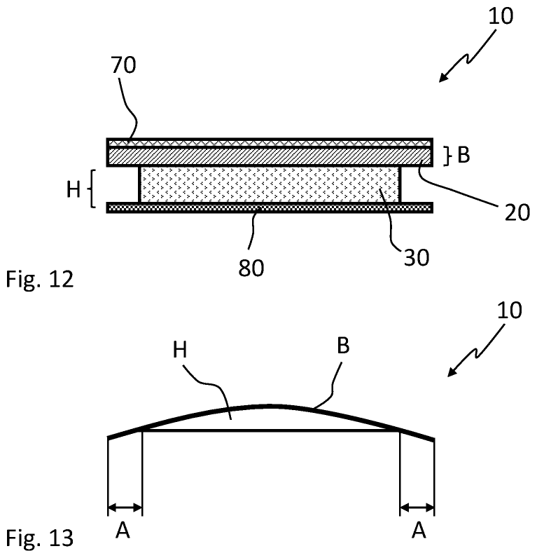

FIG. 13 shows a schematic cross sectional view of the adhesive body dressing 10. The hydrocolloid adhesive layer H has a larger thickness in the central portion of the adhesive body dressing 10 than in the peripheral portions of the hydrocolloid adhesive layer H. Thus, the thickness of the hydrocolloid adhesive layer H decreases from the central portion of the adhesive body dressing 10 towards the edges of the adhesive body dressing 10 or towards the peripheral areas A. This may provide the structure of the adhesive body dressing 10 more resistant to wear and enhance the attachment of the adhesive body dressing 10 skin during use.

The invention has been described above with reference to the examples shown in the figures. However, the invention is in no way restricted to the above examples but may vary within the scope of the claims.

1.-16. (canceled)

17. An adhesive body dressing comprising a hydrocolloid adhesive layer (H) and a backing layer (B) provided on a first side of the hydrocolloid adhesive layer (H), wherein the backing layer (B) and the hydrocolloid adhesive layer (H) are made of biodegradable polymer materials.

18. The adhesive body dressing according to claim 17, wherein the hydrocolloid adhesive layer (H) comprises:
a hydrocolloid adhesive material layer; or
a first hydrocolloid adhesive material layer and a second hydrocolloid adhesive material layer, the first hydrocolloid adhesive material layer being provided between the backing layer (B) and the second hydrocolloid adhesive material layer.

19. The adhesive body dressing according to claim 17, wherein:
the hydrocolloid adhesive material layer is made of biodegradable polymer material; or
the first hydrocolloid adhesive material layer and the second hydrocolloid adhesive material layer are made of biodegradable polymer materials.

20. The adhesive body dressing according to claim 17, wherein:
the backing layer (B) comprises a surface backing material layer provided on the first side of the hydrocolloid adhesive layer (H); or
the backing layer (B) comprises a surface backing material layer and an intermediate backing material layer, the intermediate backing material layer is provided between the surface backing material layer and the hydrocolloid adhesive layer (H).

21. The adhesive body dressing according to claim 17, wherein the backing layer (B) or the surface backing material layer comprises:
biodegradable agro-polymers; or
biodegradable biopolyesters; or
biodegradable agro-polymers and biodegradable biopolyesters.

22. The adhesive body dressing according to claim 21, wherein the backing layer (B) or the surface backing material layer comprises:
biodegradable agro-polymers; or
biodegradable agro-polymers, the biodegradable agro-polymers comprising one or more of the following: polysaccharides, proteins and lignins.

23. The adhesive body dressing according to claim 21, wherein the backing layer (B) or the surface backing material layer comprises:
biodegradable biopolyesters; or
biodegradable biopolyesters, the biodegradable biopolyesters comprising one or more of the following: polyhydroxyalkanoates, polylactides, polyglycolide, polycaprolactones, polyesteramides, polyurethane, aliphatic copolyesters and aromatic copolyesters.

24. The adhesive body dressing according to claim 21, wherein the backing layer (B) or the surface backing material layer is a film layer or a foam layer.

25. The adhesive body dressing according to claim 21, wherein the intermediate backing material layer is an intermediate elastomer layer.

26. The adhesive body dressing according to claim 21, wherein the intermediate backing material layer comprise s one or more of the following:
biodegradable agro-polymers;
biodegradable agro-polymers, the biodegradable agro-polymers comprising one or more of the following: polysaccharides, proteins and lignins;
biodegradable biopolyesters; and
biodegradable biopolyesters, the biodegradable biopolyesters comprising one or more of the following: polyhydroxyalkanoates, polylactides, polyglycolide, polycaprolactones, polyesteramides, polyurethane, aliphatic copolyesters and aromatic copolyesters.

27. The adhesive body dressing according to claim 17, wherein:
the backing layer (B) has a thickness between 0,01- to 0.1 mm; or
the backing layer (B) has preferably a thickness between 0,02-0.05 mm.

28. The adhesive body dressing according to claim 17, wherein:
the hydrocolloid adhesive layer (H) has thickness between 0,3 to 3 mm; or
the hydrocolloid adhesive layer (H) has thickness between 0.5 to 2 mm.

29. The adhesive body dressing according to claim 17, wherein the adhesive body dressing comprises a first removable covering layer provided on a second side of the hydrocolloid adhesive layer (H).

30. A method for manufacturing an adhesive body dressing having a hydrocolloid adhesive layer (H) and a backing layer (B), wherein the method comprises:
forming a backing layer (B) of a biodegradable polymer material; and
providing an adhesive hydrocolloid layer (H) on the backing layer (B), the adhesive hydrocolloid layer being made of biodegradable material.

31. The method according to claim 30, wherein the step of providing an adhesive hydrocolloid layer (H) comprises:
forming a hydrocolloid adhesive material layer; or
forming a first hydrocolloid adhesive material layer and a second hydrocolloid adhesive material layer, the first hydrocolloid adhesive material layer being provided between the backing layer (B) and the second hydrocolloid adhesive material layer.

32. A method of using a biodegradable material comprising providing a backing layer (B) of an adhesive body dressing comprising a hydrocolloid adhesive layer (H), the backing layer (B) being provided on a first side of the hydrocolloid adhesive layer (H).
